(19) United States
(12) Patent Application Publication
(10) Pub. No.: US 2012/0123375 A1
(43) Pub. Date: May 17, 2012

(54) Corbin diapers

(76) Inventor: Angelique Morgan, Philadelphia, PA (US)

(21) Appl. No.: 13/317,881

(22) Filed: Oct. 31, 2011

Related U.S. Application Data
(60) Provisional application No. 61/455,987, filed on Oct. 29, 2010.

(51) Int. Cl. A61L 15/44; A61L 15/20

(52) U.S. Cl. 604/377; 604/367

(57) ABSTRACT

The invention provides a line of disposable diapers, comprising diapers coated in a layer of soothing baby ointment, thus providing the accessories necessary for a quick diaper change when on the go or at home. Configured expressly for wear by infants who suffer diaper rash, the Corbin Diapers are designed to provide soothing relief to diaper rash symptoms such as dry and chafing skin, while sparing the parent the time consuming task of manually applying messy diaper rash ointment to the child's bottom.

### CLAIM OF PRIORITY

This patent application claims priority under 35 USC 119 (e) (1) from U.S. Provisional Patent Application Ser. No. 61/455,987 filed Oct. 29, 2010, of common inventorship herewith entitled, “Corbin Diapers.”

### FIELD OF THE INVENTION

The present invention pertains to the field of baby diaper garments, and more specifically to the field of ointment treated baby diaper garments.

### BACKGROUND OF THE INVENTION

The prior art has put forth several designs for ointment treated baby diaper garments. Among these are:

U.S. Pat. No. 6,689,932 to Benjamin Joseph Kruchoski et al describes absorbent articles with simplified compositions having good stability.

U.S. Pat. No. 6,749,840 to David John Tyrrell et al describes absorbent articles with non-aqueous compositions containing botanicals.

U.S. Pat. No. 6,756,520 to Duane Gerard Krzysik et al describes a hydrophilic composition for use in absorbent articles to enhance barrier function of the skin.

None of these prior art references describe the present invention.

### SUMMARY OF THE INVENTION

It is an object of the present invention to provide an improved ointment treated baby diaper garment.

### BRIEF DESCRIPTION OF THE DRAWINGS

FIG. 1 is an elevational angled perspective view of the device of the present invention.

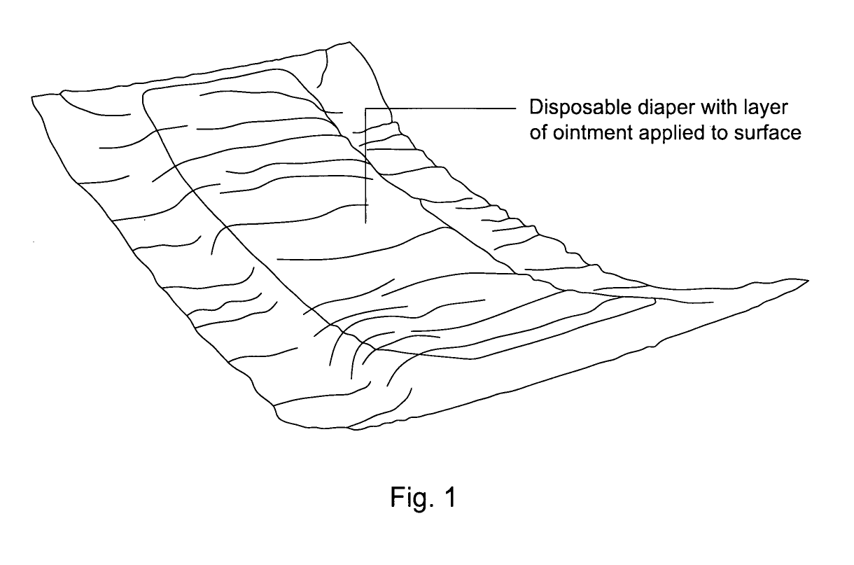

### DETAILED DESCRIPTION OF THE INVENTION

The present invention, hereinafter referred as the Corbin Diapers, are a specially designed line of disposable diapers, comprising diapers coated in a layer of soothing baby ointment, thus providing the accessories necessary for a quick diaper change when on the go or at home. Configured expressly for wear by infants who suffer diaper rash, the Corbin Diapers are designed to prevent diaper rash and to provide soothing relief to diaper rash symptoms such as dry and chafing skin, while sparing the parent the time consuming task of manually applying messy diaper rash ointment to the child's bottom. Similar in appearance to a standard infant diaper, the Corbin Diapers are manufactured of absorbent cotton-like absorbent material and backed with soft, cloth-like material and feature a stretchy trim around the leg openings. Further, the Corbin Diapers also feature a cushy, cotton-like waistband held securely closed around the child's waist by means of self affixing adhesive or hook and loop tab closures, appropriately positioned on each hip. As mentioned, the most notable aspect of the Corbin Diapers is that the interior of these diapers is coated in a layer of silky smooth diaper rash ointment. Any suitable ointment can be used. Considerations for soothing baby ointment include everything from traditional zinc based formulas, to those infused with Vitamin E, aloe, and lanolin for example. As with traditional diapers, the Corbin Diapers can be manufactured in a range of sizes for wear by newborns, infants and toddlers. Adult sizes are also contemplated. Corbin Diapers can be packaged and sold in a similar manner as traditional diapers, in a lightweight plastic bag, and can be sold in multi-count packages. As an additional consideration, Corbin Diapers can be individually packaged and labeled for sale in vending machines such as those found in public rest room facilities, providing busy parents with all the essentials necessary to care for their child when on the go.

Use of Corbin Diapers is very simple and straight forward. Consumers simply purchase Corbin Diapers as they do any other diaper, in accordance to the size and age of their child, as well as in accordance to the preference of baby ointment contained within. Next, the user simply dresses their child in the individual Corbin Diapers as they do any other disposable diaper, by placing the diaper beneath the child's bottom and pulling the front end up between the child's legs. The user then secures the diaper in place about their child's waist, via the integrated closure tabs, ensuring a comfortable and secure fit. The child then wears the Corbin Diapers per usual, throughout the course of the day or evening. During wear, the ointment which coats the interior of the diaper contacts the child's bottom, thus ensuring that the child's bottom is generously protected. Periodically, the parent simply removes the Corbin Diapers diaper, replacing it with a fresh, clean diaper. After use, the Corbin Diapers are easily discarded in any trash receptacle.

Corbin Diapers are a practical product invention which offers consumers many significant benefits and advantages. Foremost, Corbin Diapers provide parents a simple and efficient means of applying soothing baby ointment to their child's tender bottom. Consumers appreciate that each Corbin Diapers contain a generous supply of baby ointment, continuously coating the child's bottom during normal wear. Providing a silky soft and absorbent barrier between the child and moisture and waste matter found within the diaper, this self contained ointment also serves to prevent friction and heat from building up between the child's legs and the diaper's elastic banding. A practical alternative to applying cream ointment or lotion by hand, Corbin Diapers are applied quickly and in a neat manner. Another important advantage of the Corbin Diapers is found in the diaper itself. Cushiony soft and super absorbent, use of the Corbin Diapers ensures the child stays dry and comfortable throughout the course of a busy day or evening's rest. Corbin Diapers also prove a valuable commodity when traveling or on the go. Providing a fully functioning baby diaper and protective baby ointment in one convenient package, Corbin Diapers are easily tossed in a purse or glove compartment providing all of the accessories necessary for a quick change. Although designed with household consumers in mind, Corbin Diapers prove invaluable to baby sitters and other child care providers, enabling these professionals to easily care for their charges. Additionally, because this product is conveniently sold in public vending machines, consumers appreciate that Corbin Diapers are made available at restaurants, shopping malls, amusement parks and a myriad of other public facilities, providing a speedy change whenever needed.

Practical and convenient, Corbin Diapers are an innovative product invention which offers parents a neat and efficient way in which to utilize baby ointment when changing their child. Simple to use, this cleverly designed product proves invaluable for any parent and child. Affordably priced, Corbin Diapers are well received by parents and professional caregivers.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An improved ointment treated baby diaper garment, comprising: a disposable diaper wherein the surface of the diaper in contact with the skin is coated in a layer of soothing baby ointment.

2. The diaper garment of claim 1 manufactured of absorbent cotton-like material and backed with soft, cloth likematerial and feature a stretchy trim around the leg openings.

3. The diaper garment of claim 1, further comprising a cushy, cotton-like waistband held securely closed around the child's waist by means of self affixing adhesive or hook and loop tab closures, appropriately positioned on each hip.

4. The diaper garment of claim 1 wherein the ointment is selected from the group consisting of: any suitable ointment, zinc based formulas, ointments containing Vitamin E, ointments containing aloe, and ointments containing lanolin.

5. The diaper of claim 1, wherein the diapers are manufactured in a range of sizes for wear by newborns, infants, toddlers and adults.

6. The diaper of claim 1, wherein the diaper is packaged and sold in a similar manner as traditional diapers, in a lightweight plastic bag, and can be sold in multi-count packages.

7. The diaper of claim 1, wherein the diaper is individually packaged and labeled for sale in vending machines, gas stations and convenience stores.
